(19) United States
(12) Patent Application Publication
(10) Pub. No.: US 2022/0296765 A1
(43) Pub. Date: Sep. 22, 2022

(54) Photocatalytic Odor Control and Destruction Device

(71) Applicant: i-Quest Inc., Las Vegas, NV (US)

(72) Inventor: Fortunato Villamagna, Las Vegas, NV (US)

(21) Appl. No.: 17/583,174

(22) Filed: Jan. 24, 2022

Related U.S. Application Data
(60) Provisional application No. 63/163,710, filed on Mar. 19, 2021.

(51) Int. Cl. A61L 9/20

(52) U.S. Cl. CPC A61L 9/205; A61L 2209/111

(57) ABSTRACT

A photocatalytic odor control device includes an air passageway defined between an air inlet and an air outlet and configured to conduct and direct air flow, a photocatalytic plate disposed within the air passageway, the photocatalytic plate having a titanium dioxide coating incorporating metal oxide ion dopants on the surface of the plate, and a light source configured to emit an ultraviolet light onto the photocatalytic plate, the emitted ultraviolet light having a predetermined wavelength sufficient to trigger a photocatalytic reaction to generate hydroxyl free radicals and reactive oxygen species to neutralize organic compounds in the air flow.

### RELATED APPLICATION

This application claims priority to U.S. Provisional Application No. 63/163,710 filed Mar. 19, 2021.

### FIELD

The present disclosure relates to airborne odor, infestation, and microbial control devices, and more particularly, to an industrial scale photocatalytic odor control system that reduces or destroys odors, kills microbes and insect infestations, and decomposes volatile organic compounds present in the air.

BACKGROUND

Photocatalytic Oxidation (PCO) is a technology used for elimination or reduction of the level of contaminants in a fluid, such as air or water, using the chemical action of light. When ultraviolet (UV) light is used to energize a photocatalyst, the technology is more specifically termed Ultraviolet Photocatalytic Oxidation (UV-PCO).

### BRIEF DESCRIPTION OF THE DRAWINGS

FIG. 1 is a perspective view of an embodiment of a photocatalytic odor control device according to the teachings of the present disclosure;

FIG. 2 is a schematic diagram of an embodiment of a photocatalytic odor control device according to the teachings of the present disclosure;

FIG. 3 is a simplified block diagram of an embodiment of the communication and control network of a photocatalytic odor control devices according to the teachings of the present disclosure;

FIG. 4 is a flowchart of an embodiment of a photocatalytic process to remove organic based odors and contaminants according to the teachings of the present disclosure;

FIG. 5 is an illustration of the photocatalytic process according to the teachings of the present disclosure;

FIG. 6 is a flowchart of an embodiment of the flame spray coating process according to the teachings of the present disclosure; and

FIG. 7 is an illustration of an embodiment of the flame sprayer coating mechanism according to the teachings of the present disclosure.

### DETAILED DESCRIPTION

The photocatalytic odor control device 100 described herein uses a novel thin film composition of a semiconductor material, such as titanium dioxide, deposited or otherwise coated in a specific and novel way on a substrate that may comprise a ceramic or a metal (such as aluminum and stainless steel).

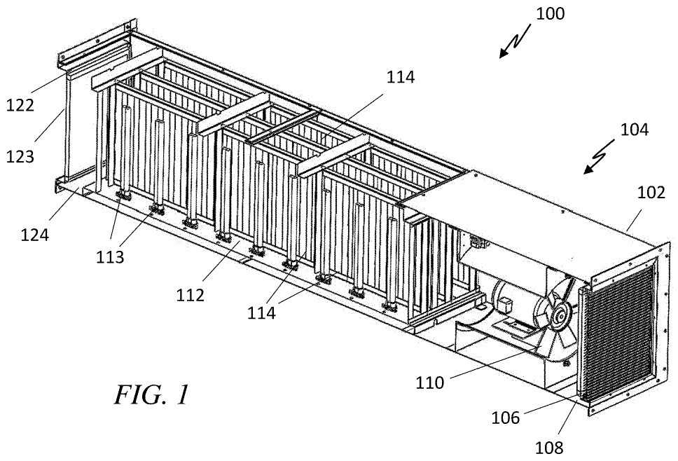

FIG. 1 is a perspective view of a photocatalytic odor control device 100 with some of the housing panels removed to show the internal structures that may be used in industrial and commercial applications. The device 100 preferably includes a structurally sound housing 102 fabricated of a metal such as stainless steel. In a first compartment 104 of the housing 102, one or more particulate and/or optical filters 106 are positioned to remove particulates and other contaminants (e.g., dust, dust mites, pollen, dander, mold) from the flow of air entering the housing 102 via an inlet (not explicitly shown). The particulate filters 106 may include high efficiency particulate air (HEPA) and/or MERV-rated (minimum efficiency reporting value) filters. The first compartment 104 may further include a debris tray 108 positioned proximately to the first particulate filter screen 106 to collect debris and particulates that are blocked by the first filter screen 106 and drops from the airflow. Situated behind the particulate filters 106 in the first compartment 104 is a fan 110 that is operable to draw air into the housing 102 via the inlet and push the air into a first air treatment chamber 112 that functions as the photocatalytic chamber. The use of an electrical mechanism to move air and create airflow is optional. Alternatively, airflow within the housing 102 may be created by utilizing natural convection.

Located within the photocatalytic chamber 112 is a plurality of photocatalytic plates 114 arranged in a predetermined pattern to provide maximum exposure to airflow. In the embodiment shown in FIG. 1, the photocatalytic plates 114 are arranged in parallel with one another and form multiple parallel air passageways in the chamber 112. The plates 114 have a titanium dioxide-coated surface and a generally corrugated profile that helps to maximize the active photocatalytic surface area of the plates. The corrugated surface also helps to promote turbulence as the air flows over the plates. One or more ultraviolet (UV) light emitting devices such as UV LEDs (light emitting diodes) are disposed in the photocatalytic chamber 112 so that all surface areas of the photocatalytic plates 114 are exposed to ultraviolet light. To ensure maximum exposure, at least one light emitting device is positioned in each aisle between rows of the plates to illuminate the corrugated surfaces. The UV light emitting devices 113 may project UV light upward from the floor of the chamber 112 and/or downward from the ceiling of the chamber 112.

The photocatalytic chamber 112 is preferably shielded by one or more louvers 122 that provides an optical screen that prevents UV light from escaping the photocatalytic chamber 112. At least one coarse filter 123 is further provided at the exit. Exiting the photocatalytic chamber 112, the processed airflow enters a second and optional air treatment chamber 124 where a manganese-based (MnO₂) catalyst is positioned. The manganese-based catalyst may be in any suitable form, such as pellets, and it is capable of neutralizing any remaining ozone (O₃) that remains in the airflow. After leaving the second chamber 124, the treated air exits the housing 102.

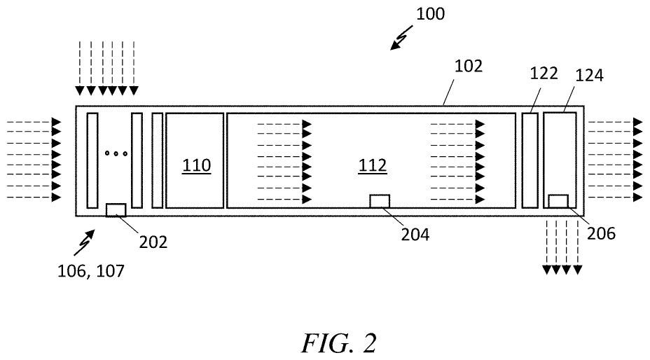

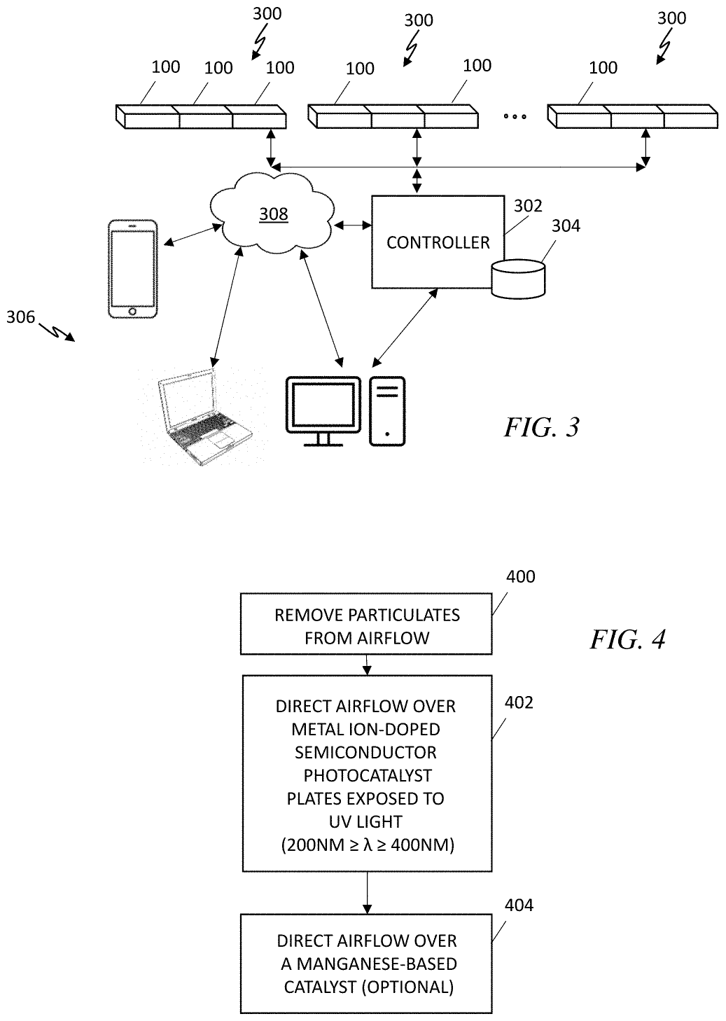

FIG. 2 is a schematic diagram of the photocatalytic odor control device 100. The orientation, alignment, and configuration of the air inlet, the airflow path(s), and the air outlet of the housing 102 may not be in linear alignment. For example, the inlet and/or the outlet may be positioned so that the inflow and/or outflow is perpendicular to the airflow inside the photocatalytic chamber 112. Two or more photocatalytic odor control systems 100 may be coupled in series such as shown in FIG. 3, and an installation may include multiple series of photocatalytic odor control systems 300 installed at various points inside a facility and in communication with a controller 302 via a wired or wireless computer network using a suitable communication protocol. Depending on the type of communication link that connect the controller 302 and the odor control systems 100, the appropriate interface devices such as transmitters/receivers are incorporated to enable data exchange. The photocatalytic odor control devices 100 may incorporate various sensors 202-206 (FIG. 2) that measure or sense a number of parameters in the system, such as temperature, humidity, air speed, oxygen, carbon dioxide, particulates, and pollutants at one or more points in the housing, and these measurement/sensor data are transmitted to the controller 302 and stored in a database 304. The controller 302 and the database 304 may further be in communication with one or more user computing devices 306 via a wired or wireless computer network, which may include the cellular network and the internet 308. The controller 302 may include computer software that controls and monitors the operation of the photocatalytic odor control systems, including adjusting any of the operating parameters such as humidity, temperature, airspeed, UV light intensity, etc.

FIG. 4 is a simplified flowchart of an embodiment of the photocatalytic odor control process according to the teachings of the present disclosure. In step 400, large particle size particulates are removed from the airflow. In step 402, the airflow is directed over one or more titanium oxide-based photocatalytic plate that is exposed to UV light of a certain intensity where the wavelength of the light is preferably between 200 nm and 400 nm. The UV light causes a photocatalytic reaction that neutralizes odors, and purifies and sterilizes the air. In step 404, the airflow is then treated with a manganese-based catalyst that removes ozone that may remain after the photocatalytic reaction. The treated air is then returned to the facility.

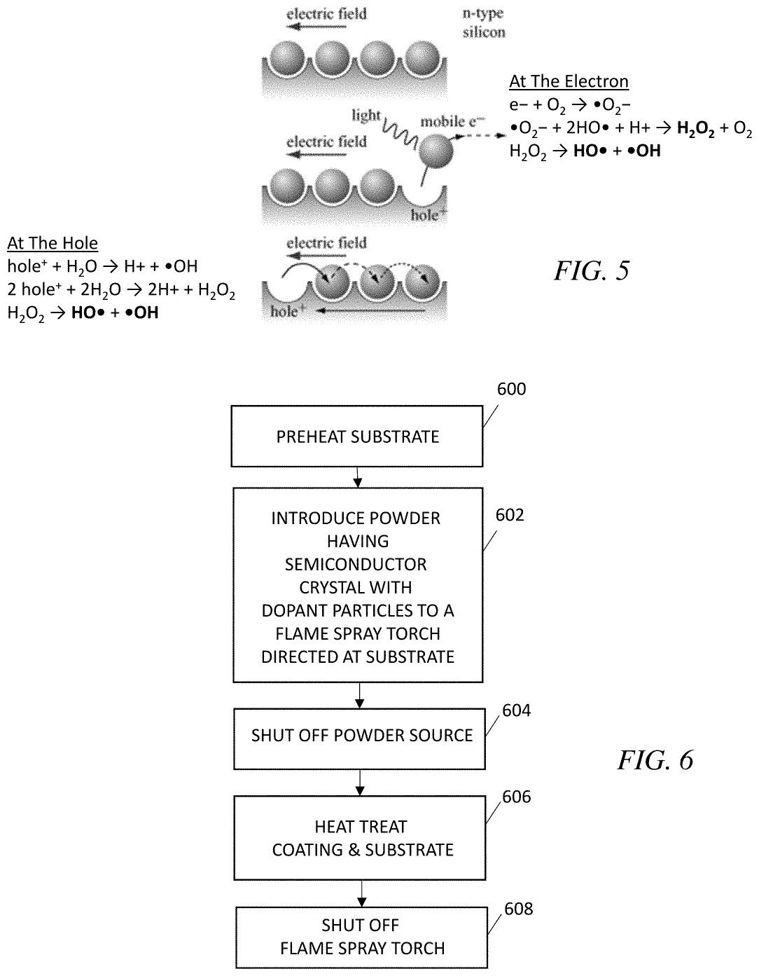

As shown in FIG. 5, when UV light shines on the titanium dioxide-coated substrate of the plates 114, electrons are energized and released from its surface. The electrons enter the conduction band and leave holes behind. The electrons interact with oxygen (O₂) and water vapor (H₂O) in the air, as well as nitrogen and any carbon-based compound in the air, breaking them up into reactive oxygen species like ozone, singlet oxygen, peroxide radicals, and hydroxyl free radicals (OH·), all of which are highly reactive and short-lived uncharged molecules. Other examples for the radicals include Superoxide (O·—₂), Oxygen radical (O··₂), Hydroxyl (OH) as noted above, Alkoxyradical (RO·), Peroxyl radical (ROO), Nitric oxide (nitrogen monoxide) (NO^{·}) and nitrogen dioxide (N·₂). The high reactivity of these radicals is due to the presence of one unpaired electron which tends to donate it or to obtain another electron to attain stability. These small agile molecules are reactive agents that “attack” bigger organic (carbon-based) pollutant molecules in the air, breaking apart their chemical bonds, and turning them into harmless substances such as carbon dioxide (CO₂) and water (H₂O). This is an oxidation process that is also described as photocatalytic oxidation or PCO. The novel combination outlined in this disclosure optimizes the formation of the reactive oxygen species and the type of species that are generated as a result of the photocatalytic process.

The photocatalytic reaction employed by the odor control device 100 involves photocatalytic plates 114 made of semiconductors with a sufficiently wide band gap energetic enough to activate water or surface hydroxyl ions thus creating .OH radicals that eliminate organic contaminants. These semiconductor materials include, but are not limited to, titanium dioxide

(TiO₂), zirconium dioxide (ZrO₂), zinc oxide (ZnO), calcium titanate (CaTiO₃), tin (stannic) dioxide (SnO₂), molybdenum trioxide (MoO₃), and the like. Of this group, titanium dioxide (TiO₂) is the preferred photocatalyst because of its chemical stability, relatively low cost, and electronic band gap that is suitable for photoactivation by UV light. The novel semiconductor composition/matrix used in this application provides a very specific bandgap, allowing more efficient formation of the reactive oxygen species.

There are two important types of titanium dioxide (TiO₂)—rutile titanium dioxide and anatase titanium dioxide. The key difference among them is in their appearance. Anatase titanium dioxide is colorless, whereas rutile titanium dioxide is usually found in a dark red appearance. Rutile titanium dioxide is optically positive, whereas anatase titanium dioxide is optically negative. Rutile titanium dioxide has highly stable and is the most common type of titanium dioxide usually found in metamorphic and igneous rocks. Rutile titanium dioxide has a crystalline structure with a tetragonal unit cell having oxygen anions and titanium cations. The coordination number of titanium cations (Ti⁺⁴) is 6, while the coordination number of oxygen anion (O²⁻) is 3. Some vital properties of rutile titanium dioxide nanoparticles include greater dispersion, higher birefringence, and greater refractive index (RI) at visible wavelengths.

Rutile titanium dioxide has many useful applications including the manufacturing of metallic titanium and titanium dioxide pigments. It is also used for manufacturing plastics, papers, and paints. The finely pulverized form of titanium dioxide is white in color. Furthermore, the nanoparticles of rutile titanium dioxide have the ability to absorb UV rays and are transparent to visible light. That is the reason that they are used in the manufacturing of cosmetics.

The application described herein involves a suspension layer, or agglomerate of semiconductor nanoparticles, is irradiated with UV light that causes excitation of an electron from the valence band to the conduction band. This results in the formation of an oxidizing site (hole) in the valence band and a reducing site (electron) in the conduction band. Thus, organic compounds, cell walls, and molecules in general, are oxidatively degraded into harmless reaction products. Direct use of solely semiconductors like titanium dioxide is generally limited by its weak light absorption properties, large bandgap, and low oxidation efficiency. Therefore, photocatalytic oxidation procedures involving nanoparticles of TiO₂ has been modified in many ways to improve its adsorption ability and efficiency. For example, surface-fluorinated TiO₂ and ZnO/TiO₂ nanocomposite films have been applied as photocatalysts. Further, adding an electron scavenger like Ce4+ also improves the efficiency of holes. Various other modifications have been proposed like use of molecular sieve 4A-TiO2-K₂Cr₂O₇ system as a sensor and in-situ surface modification of TiO₂ with 5-sulfosalicylic acid and KMnO4 catalysts in COD analysis.

The photocatalytic odor control technology is especially adapted to neutralize organic molecules present in the air that contribute to nuisance odors that result from certain agricultural, industrial, and commercial processes and activities such as food processing, waste processing, wastewater treatment, landfills, composting, animal feed manufacturing, breweries, distilleries, spice production, and cannabis operations. Further, photocatalytic air purification may be used in high occupancy or even residential applications to address volatile organic compounds (VOCs) that are potentially harmful gases emitted by a wide array of common household products. They are a primary cause of indoor air quality problems in the home. Young children, the elderly and people with respiratory problems may be more affected by VOC exposure than others. According to the U.S. Environmental Protection Agency (EPA), concentrations of many VOCs are up to ten times higher indoors than outdoors—regardless of whether the home is located in a rural or highly industrialized area. Other sources of odor include bacteria, virus, mold, mildew, yeast, microbes, smoke, allergens (e.g., dust, perfumes, pet dander, and pollen), and pest infestations (e.g., pheromones and waste).

The biggest advantage that photocatalytic odor control systems have over other air-cleaning technologies is that instead of simply trapping pollutants in a filtration material that still needs to be replaced and disposed of, the photocatalytic process employed by the odor control device 100 completely transform the harmful and odorous chemical molecules in the air and effectively destroys them.

### THERMAL COATING PROCESS

As stated above, the photocatalytic odor control device 100 includes one or more plates 114 that has a substrate with a thin layer of semiconductor material (e.g., titanium dioxide) coating. In an exemplary embodiment, an elevated temperature coating process is used to deposit the titanium dioxide onto the substrate.

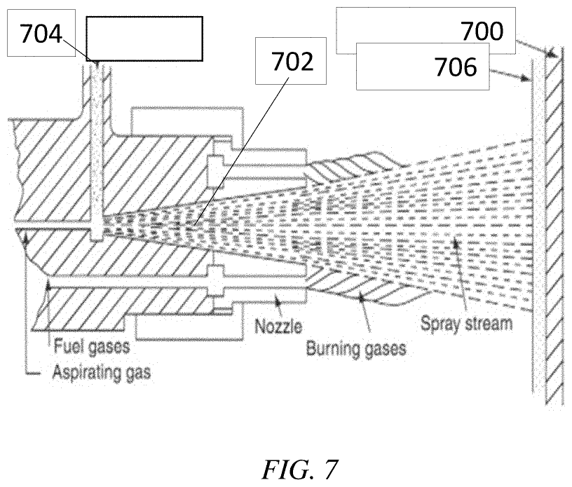

Referring to FIGS. 6 and 7, the coating process uses a thermal spraying device. Flame spraying and other thermal coating techniques such as high velocity oxygen fuel (HVOF) coating use the chemical energy of combusting fuel gases to generate heat and consequently accelerate the molten particles toward the substrate. Oxygen acetylene carrier gasses and their combustion products are the most common, using acetylene (C₂H₂) as the main fuel in combination with oxygen to generate the highest combustion temperature of approximately 3000° C. Other gases in use are propane (C₃H₈), propylene (C₃H₆), hydrogen (H₂), and ethane (C₂H₄). The deposited material may be introduced as a powder, wire, or rod axially through the rear of the deposition system. An exemplary embodiment of the preferred thermal coating process involves first preheating the surface of a substrate or base material 700 with a flame spray torch 702 (step 600), then blowing a powder 704 through the flame from the spray torch 702 (step 602). The powder 704 is a mixture of semiconductor (e.g., titanium dioxide) crystals with certain dopants of a certain average particle size. The flame spray torch 702 partially melts the powder and as the molten powder contacts the surface of the substrate 700, it solidifies and forms a thin layer 706 on the surface. When the deposited layer 706 is of a sufficient thickness, the powder source is shut off (step 604). The flame spray torch remains on to continue to heat treat the coating (706) and the substrate (step 606). Post-deposition thermal treatment of the substrate surface is another novel aspect of the coating process. The flame spray torch 702 is then shut off after completion (608).

The doped semiconductor powder can be fed into flame spray torch by a carrier gas or by gravity. Gravity-fed devices may use powder canisters or bottles mounted directly to, and on top of, the torch. Powder feed rate is controlled by a pinch valve that meters the powder into the body of the torch, where it is aspirated by the gases flowing through the torch. Carrier-gas-fed units use externally mounted powder feeders. External powder feeders use a carrier gas (typically nitrogen, air is also used) stream to transport the powder from the feeder through a hose to the spray torch. Wire- and rod-fed devices use air turbines built into the torch that power the drive rolls, which pull feedstock from the source and push it through the nozzle.

In this thermal coating process, the feedstock materials are utilized in the molten state at specific temperatures based on total composition. The carrier gas composition as well as the cooling and annealing rate of the post-deposition substrate surface are also novel elements of this application. The feedstock materials are molten by the spray torch and the particles/droplets accelerate toward the substrate surface by the expanding gas flow and in some cases also by air jets.

In the deposition processes, the fuel/oxygen ratio and total gas flow rates can be adjusted to produce the desired thermal output needed to melt the specific feedstock material. Optional air jets, downstream of the combustion zone, may also further adjust the thermal profile of the flame. In flame spray processes, the fuel/oxygen ratio and total gas flow rates are adjusted to produce the desired thermal output needed to melt the specific feedstock material. Optional air jets, downstream of the combustion zone, may also further adjust the thermal profile of the flame. Spray gas speeds typically are below 100 m s⁻¹, generating particle speeds up to approximately 80 m s⁻¹ before impact.

Forming a coating with a post-heat treatment by sintering/fusing can be carried out to obtain dense coatings with metallurgical bonding (diffusion bonds). This process consists of two separate stages, spraying and post-fusion, and is clearly different from other conventional processes, which are one-step processes and in which the coating adherence to the substrate material is mainly of the type of mechanical anchoring and bonding to the substrate surface asperities created by grit blasting of the surface prior to thermal coating. These types of coatings are homogeneous with good bonding and adhesion strength, 350-500 MPa. Because of the high fusing temperature (approximately 1050° C.), there is a risk of deformation.

Titania (TiO₂) is a white oxide ceramic and comes in three crystalline forms: rutile with a tetragonal structure, anatase also with a tetragonal structure, and brookite with an orthorhombic structure. The preferred crystalline structure is a modified anatase, which can be modified by adding certain dopants or impurities. Doping of TiO₂ with various elements increases its photocatalytic activity due to the formation of new energy levels near the conduction band. Photocatalysis involving titanium dioxide is a heterogeneous process in which the surface of the catalyst plays an important role. The structural properties of TiO₂ are influenced by the spraying conditions, the doping concentration, and the dopants. The incorporation of dopants leads to the distortion of the TiO₂ crystal lattice, thus changing its surface characteristics, and a decrease in the energy band gap. For example, the introduction of aluminum (Al) and copper (Cu) increases the photocatalytic activity by as much as 50% while doping with Molybdenum (Mo) and Tungsten (W) increases the activity by 75%. The dopant concentration can be in the range of 100 ppm and 500,000 ppm.

Upon doping with copper, clusters of copper oxide are formed on the surface of TiO2, which can also take part in the photocatalysis. The use of Cu is a more affordable and cheaper alternative to elements such as silver and gold, as well as platinum group metals. There also has been increasing interest in the use of trivalent metals (Al, Niobium—Nb) as doping materials as they improve the electrical, optical, and structural characteristics of titanium dioxide. Doping of

Al, Cu, Mo, or W leads to a narrowing of the band gap of TiO₂, which increases its photocatalytic activity upon irradiation with visible light.

The introduction of Al³⁺ into the crystal lattice of titanium dioxide leads to the appearance of oxygen vacancies, which increases the photocatalytic activity. In the cases of transition metals of molybdenum and tungsten, not only Mo⁶⁺ and W⁶⁺ ions but also Mo⁴⁺, Mo⁵⁺, W⁴⁺, W⁵⁺, as well as Ti³⁺ ions, which also take part in photocatalytic processes, can be present on the powder surface.

XRF (X-ray fluorescence) is a non-destructive analytical technique used to determine the elemental composition of materials. XRF analyzers determine the chemistry of a sample by measuring the fluorescent (or secondary) X-ray emitted from a sample when it is excited by a primary X-ray source. Each of the elements present in a sample produces a set of characteristic fluorescent X-rays (“a fingerprint”) that is unique for that specific element, which is why XRF spectroscopy is an excellent technology for qualitative and quantitative analysis of material composition.

Photocatalytic activity of TiO₂ increases as the particle size of TiO₂ is decreased, especially when the particle size is less than 30 nm. The half-life (t0.5) of the photocatalytic degradation of MB also decreased as the particle sizes of TiO₂ decreased. The preferred titanium dioxide particles sizes include: 1 μm, 5 μm, 50 μm, 100 μm, 200 μm, and 500 μm.

### OZONE MITIGATION

One potential output from conventional photocatalytic odor control systems is ozone. The U.S. Occupational Safety and Health Administration (OSHA) requires that the indoor “threshold limit value” (TLV) of an eight-hour exposure be limited to 0.1 part per million (ppm). The photocatalytic odor control system described herein prevents the direct formation of ozone by using the precise frequency of the UV light that activates the TiO₂ to destroy ozone.

The crystalline structure (Anatase)—particle size (˜1 micron)—and dopants are selected to create the band gap in the TiO₂ of 3.2 eV or greater, which corresponds to a wavelength of 388 nm or lower. The larger the band gap eV value, the greater the energy, and the lower the wavelength. Any wavelength less than 388 nm will activate the TiO₂. UV light having a wavelength between 240 nm and 388 nm will break down ozone molecules to form oxygen. As long as UV light with a wavelength above 240 nm is used for the photocatalytic process, the dual functions of activating the TiO₂ and neutralizing the ozone are achieved. In an exemplary embodiment, UV light having a wavelength of between 240 nm and 388 nm is used to activate the titanium dioxide and also destroy the resultant ozone. In an exemplary embodiment, the method includes irradiating the photocatalyst with an ultraviolet light having a wavelength between 240 nm and 388 nm at an intensity of greater than 1 W/cm².

In the event ozone is formed due to catalyst breakdown, fouling, etc., an optional secondary process can be used to treat the air before it exits the photocatalytic odor control device 100. Manganese dioxide-based catalysts may be used for ozone destruction since it is highly effective in destroying ozone at ambient room temperature. These catalysts show very high ozone destruction efficiencies even in high humidity applications. Manganese dioxide catalysts require only a 0.36 second residence time, which means relatively small catalyst volumes are needed, making the catalytic system with a manganese dioxide-based catalyst very cost-effective. The inlet concentration of ozone does not affect the amount of catalyst required or the design of the system. Ozone concentrations ranging from a few ppm to well over 100K ppm can be effectively controlled by the use of this catalyst in the second treatment chamber 124.

### DOPANTS

Rutile is a direct bandgap semiconductor, meaning that under UV excitation (E>3.0 eV) electrons can easily be promoted from the rutile valence band (+2.3 V vs NHE) to the conduction band (−0.7 V vs NHE). However, fast electron-hole pair recombination also occurs. The net result is that there are few charge carriers available for photoreactions at the surface of rutile.

Anatase is an indirect bandgap semiconductor (VB +2.7 V vs NHE, CB −0.5 V versus NHE) meaning that both the charge separation and recombination under UV excitation (E>3.2 eV) are slower in anatase than in rutile. The slower recombination rate allows more electrons and holes generated in the bulk to reach to the anatase surface and participate in photoreactions. Accordingly, anatase will generally be a better photocatalyst than rutile due to the increased number of charge carriers (electrons and holes) reaching the surface of anatase.

This discussion neglects surface area effects, which are also important when comparing the relative activities of anatase and rutile. It is important to normalize reaction rates against exposed surface area when comparing anatase and rutile photocatalysts.

The effect of 5 MeV Cu++ ions irradiation on structural and optical properties of Anatase TiO₂ nanoparticles (TiO₂-NPs) is investigated. For this purpose, TiO₂-NPs are irradiated with different Cu⁺⁺ ions fluences, ranging from 1×10¹⁵ to 1×10¹⁶ ions/cm² at room temperature. XRD results confirm the Ti₃O₇ phase appear at the dose of 5×10¹⁵ ions/cm² and peak intensity of Ti₃O₇ phase gradually increases with an increase of Cu⁺⁺ ions irradiation dose. At the dose of 1×10¹⁶ ions/cm² TiO₂ anatase phase are transformed to rutile phase. Same observations are confirmed from Raman spectroscopy. High resolution transmission electron microscopy (HRTEM) reveals that morphology converted into wavy shape and crystal structure doped with increased Cu ion irradiation dose to form vacancy loops and interstitial loops. Scanning electron microscopy (SEM) shows that TiO₂-NPs have been fused to form a cluster of nanoparticles at high Cu ion beam dose, while bandgap of TiO2-NPs reduces from 3.19 eV to 2.96 eV as a function of Cu⁺⁺ irradiation fluence. These phase transformations and crystal damage are the responsible for optical bandgap reduction. The mechanism for the currently observed phase transformation of TiO₂ and coalescence of TiO₂-NPs are discussed in term of thermal spikes model.

Rutile is a direct bandgap semiconductor, meaning that under UV excitation (E>3.0 eV) electrons can easily be promoted from the rutile valence band (+2.3 V vs NHE) to the conduction band (−0.7 V vs NHE). However, fast electron-hole pair recombination also occurs. The net result is that there are few charge carriers available for photoreactions at the surface of rutile.

Anatase is an indirect bandgap semiconductor (VB +2.7 V vs NHE, CB −0.5 V versus NHE) meaning that both the charge separation and recombination under UV excitation (E>3.2 eV) are slower in anatase than in rutile. The slower recombination rate allows more electrons and holes generated in the bulk to reach to the anatase surface and participate in photoreactions. Accordingly, anatase will generally be a better photocatalyst than rutile due to the increased number of charge carriers (electrons and holes) reaching the surface of anatase. This discussion neglects surface area effects, which are also important when comparing the relative activities of anatase and rutile. It is important to normalize reaction rates against exposed surface area when comparing anatase and rutile photocatalysts.

The effect of 5 MeV Cu++ ions irradiation on structural and optical properties of Anatase TiO₂ nanoparticles (TiO₂-NPs) is investigated. For this purpose, TiO₂-NPs are irradiated with different Cu⁺⁺ ions fluences, ranging from 1×10¹⁵ to 1×10¹⁶ ions/cm² at room temperature. XRD results confirm the Ti₃O₇ phase appear at the dose of 5×10¹⁵ ions/cm² and peak intensity of Ti₃O₇ phase gradually increases with an increase of Cu++ ions irradiation dose. At the dose of 1×16¹⁶ ions/cm² TiO₂ Anatase phase were transformed to Rutile phase. Same observations are confirmed from Raman spectroscopy. High resolution transmission electron microscopy (HRTEM) reveals that morphology converted into wavy shape and crystal structure doped with increase Cu ion irradiation dose to form vacancy loops and interstitial loops. Scanning electron microscopy (SEM) shows that TiO₂-NPs have been fused to form a cluster of nanoparticles at high Cu ion beam dose, while bandgap of TiO₂-NPs reduces from 3.19 eV to 2.96 eV as a function of Cu⁺⁺ irradiation fluence. These phase transformations and crystal damage are the responsible for optical bandgap reduction. The mechanism for the currently observed phase transformation of TiO₂ and coalescence of TiO₂-NPs are discussed in term of thermal spikes model.

It is contemplated that in addition to a stand-alone air purification system equipped with fans and other devices that circulate air, the UV light and titanium dioxide-coated plates may be designed and packaged as an air purification module that can be retrofitted or installed to work with conventional air handlers, air conditions, and other systems through which air passes in a facility or building.

The features of the present invention which are believed to be novel are set forth below with particularity in the appended claims. However, modifications, variations, and changes to the exemplary embodiments described above will be apparent to those skilled in the art, and the photocatalytic odor control system described herein thus encompasses such modifications, variations, and changes and are not limited to the specific embodiments described herein.

What is claimed is:

1. A photocatalytic odor control device comprising:
an air passageway defined between an air inlet and an air outlet and configured to conduct and direct air flow;
a photocatalytic plate disposed within the air passageway, the photocatalytic plate having a titanium dioxide coating incorporating metal oxide ion dopants on the surface of the plate; and
a light source configured to emit an ultraviolet light onto the photocatalytic plate, the emitted ultraviolet light having a predetermined wavelength sufficient to trigger a photocatalytic reaction to generate hydroxyl free radicals and reactive oxygen species to neutralize organic compounds in the air flow.

2. The device of claim 1, further comprising a particulate filter positioned in the air passageway to remove particulates from air flowing within the passageway prior to being in contact with the photocatalytic plate.

3. The device of claim 1, wherein the light source is configured to emit ultraviolet light having a wavelength between 240 nm and 388 nm.

4. The device of claim 1, wherein the photocatalytic plate has a corrugated profile and has the titanium dioxide coating incorporating metal oxide ion dopants on both sides thereof, wherein the photocatalytic plate is oriented in parallel alignment with the air flow and the coating on both sides of the plate is exposed to the ultraviolet light.

5. The device of claim 1, further comprising a fan configured to move air in the air passageway.

6. The device of claim 1, wherein the metal ion dopants are selected from the group consisting of Silver (Ag), aluminum (Al), gold, (Au), calcium (Ca), cadmium (Cd), cobalt (Co), copper (Cu), molybdenum (Mo), platinum (Pt), palladium (Pd), tin (Sn), tungsten (W), zinc (Zn), and zirconium (Zr).

7. The device of claim 1, wherein the titanium dioxide coating further comprises semiconductor selected from the group consisting of zirconium dioxide (ZrO₂), zinc oxide (ZnO), calcium titanate (CaTiO₃), tin (stannic) dioxide (SnO₂), and molybdenum trioxide (MoO₃).

8. The device of claim 1, wherein the photocatalytic plate is fabricated from a metal plate and the coating is deposited on the surface of the metal plate by a flame spray process of:
pre-heating the metal plate;
feeding a powder comprising semiconductor microscopic titanium dioxide (TiO2) crystals and metal ion dopants into a flame spray torch directed at a surface of the metal plate, thereby depositing molten powder and forming the coating on the surface of the plate;
stop feeding the powder after a predetermined thickness of the coating has been formed;
continue heating the metal plate; and
allowing the metal plate to cool.

9. The device of claim 1, further comprising an optical screen configured to prevent the ultraviolet light from exiting the air passageway.

10. The device of claim 1, further comprising a plurality of photocatalytic plates having semiconductor coating incorporating metal ion dopants arranged within the air passageway.

11. The device of claim 1, wherein the semiconductor coating comprises anatase titanium dioxide and rutile titanium dioxide.

12. The device of claim 11, wherein the ratio of anatase titanium dioxide to rutile titanium dioxide is greater than 1.

13. The device of claim 11, wherein the ratio of anatase titanium dioxide and rutile titanium dioxide is at least 6:1.

14. The device of claim 1, further comprising a manganese-based catalyst disposed within the air passageway being exposed to the air flow prior to exiting the air outlet.

15. The device of claim 1, further comprising:
a controller; and
at least one sensor in communication with the controller and configured to measure and transmit thereto a parameter selected from the group consisting of temperature, humidity, air speed, oxygen, carbon dioxide, particulates, and pollutants.

16. The device of claim 1, further comprising a housing defining the air inlet, air outlet, and the air passageway connecting the air inlet and the air outlet.

17. The device of claim 1, comprising a plurality of photocatalytic plates arranged in parallel alignment with air flow within the air passageway, each photocatalytic plate having at least one reactive surface with a semiconductor coating incorporating metal ion dopants.

18. A photocatalytic plate for use in an odor control device, the photocatalytic plate comprising:
a metal substrate having a non-planar profile;
a reactive coating of a semiconductor oxide with metal oxide ion dopants formed on the substrate, the reactive coating being deposited onto the substrate by a flame spray process having the steps of:
pre-heating the metal substrate;
feeding semiconductor microscopic crystals and metal oxide ion dopants into a flame spray torch directed at a surface of the metal substrate;
stop feeding the powder after a predetermined thickness of the coating has been formed;
continue heating the metal substrate; and
allowing the meatal substrate to cool.

19. The photocatalytic plate of claim 18, wherein the reactive coating generates hydroxyl free radicals and reactive oxygen species when exposed to ultraviolet light having a wavelength between 200 nm and 400 nm.

20. The photocatalytic plate of claim 18, wherein the substrate has a corrugated surface profile.

21. The photocatalytic plate of claim 18, wherein the metal oxide ion dopants are selected from the group consisting of Silver (Ag), aluminum (Al), gold, (Au), calcium (Ca), cadmium (Cd), cobalt (Co), copper (Cu), molybdenum (Mo), platinum (Pt), palladium (Pd), tin (Sn), tungsten (W), zinc (Zn), and zirconium (Zr).

22. The photocatalytic plate of claim 18, wherein the semiconductor oxide is selected from the group consisting of titanium dioxide (TiO₂), zirconium dioxide (ZrO₂), zinc oxide (ZnO), calcium titanate (CaTiO₃), tin (stannic) dioxide (SnO₂), and molybdenum trioxide (MoO₃).

23. The photocatalytic plate of claim 18, wherein the reactive coating comprises anatase titanium dioxide and rutile titanium dioxide, where the ratio of anatase titanium dioxide and rutile titanium dioxide is at least 6:1.

24. A method of fabricating a photocatalytic odor control device:
fabricating a photocatalytic component using a flame spray process having the steps of:
pre-heating a metal plate having a non-planar profile;
feeding materials including titanium dioxide and metal oxide ion dopants into a flame spray torch directed at a surface of the metal plate and forming a coating thereon;
stop feeding the materials after a predetermined thickness of the coating has been formed;
continue heating the metal plate; and
allowing the meatal plate to cool.

25. The method of claim 25, wherein feeding the materials comprises feeding a powder including semiconductor microscopic crystals selected from the group consisting of titanium dioxide (TiO2), zirconium dioxide (ZrO2), zinc oxide (ZnO), calcium titanate (CaTiO3), tin (stannic) dioxide (SnO2), and molybdenum trioxide (MoO3), and feeding the powder further including metal oxide ion dopants selected from the group consisting of Silver (Ag), aluminum (Al), gold, (Au), calcium (Ca), cadmium (Cd), cobalt (Co), copper (Cu), molybdenum (Mo), platinum (Pt), palladium (Pd), tin (Sn), tungsten (W), zinc (Zn), and zirconium (Zr).
